(19) United States
(12) Patent Application Publication
(10) Pub. No.: US 2003/0185779 A1
(43) Pub. Date: Oct. 2, 2003

(54) Hair care kits and heating devices for warming hair care compositions

(75) Inventors: Arata Mitsumatsu, Nishinomiya-shi (JP); Akihiko Kawauchi, Nishinomiya-shi (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/383,441

(22) Filed: Mar. 7, 2003

Related U.S. Application Data
(63) Continuation of application No. PCT/US00/24709, filed on Sep. 8, 2000.

(51) Int. Cl. A61K 7/06

(52) U.S. Cl. 424/70.1

(57) ABSTRACT

Disclosed is hair care kits used for warming a hair care composition before the composition is applied to the hair. One of the kits comprises (i) an aqueous hair care composition and (ii) a heat generating agent which generates a heat by mixing with the aqueous care composition, wherein the heat generating agent is isolated from the aqueous hair care composition. One of the heating devices comprises a heating source comprising (i) a heat generating agent which generates a heat by mixing a reacting means, and (ii) a reacting means.

### CROSS REFERENCE TO RELATED APPLICATION

The application is a continuation of International application PCT/US00/24709 (Case AA497F) filed on Sep. 8, 2000.

### TECHNICAL FIELD

The present invention relates to a hair care kit and a heating device which are used for warming a hair care composition.

### BACKGROUND

A variety of hair care products have been used to the hair. For example, hair shampoo products are used for cleaning the hair by removing excess soil and sebum; hair conditioning products are used for providing various conditioning benefits such as moisturized feel, softness, and static control to the hair; hair styling products are used for setting hair style and/or maintaining hair style; hair color products are used for changing hair color and/or maintaining hair color; and hair growth products are used for encouraging hair growth.

The efficacy of hair care products are changed by various factors, for example, amount of hair care products applied, how long hair care products are applied on the hair, temperatures of hair care products, the way of applying hair care products to the hair, and so on. Thus, it may not be easy to obtain expected efficacy from hair care products.

Based on the foregoing, there remains a desire for obtaining enhanced efficacy from hair care products, i.e., a desire for obtaining improved benefits from hair care products, for example, obtaining improved cleansing benefits from hair shampoo products, and obtaining improved hair conditioning benefits such as moisturized feel, softness, and static control from hair conditioning products.

None of the existing art provides all of the advantages and benefits of the present invention.

### SUMMARY

The present invention is directed to a hair care kit (hereinafter “Kit A”) comprising an aqueous hair care composition and a heat generating agent which generates a heat by mixing with the aqueous hair care composition, wherein the heat generating agent is isolated from the aqueous hair care composition.

The present invention is also directed to a hair care kit (hereinafter “Kit B”) comprising:

(i) a hair care composition;

(ii) a heat generating agent which generates a heat by reaction with a reacting means; and

(iii) a reacting means;

wherein the hair care composition, the heat generating agent, and the reacting means are isolated respectively.

The present invention is further directed to a heating device, used for warming a hair care composition, comprising a heating source.

These and other features, aspects, and advantages of the present invention will become evident to those skilled in the art from a reading of the present disclosure.

The present invention provides hair care kits and heating devices, which are used for warming hair care compositions. It is believed that; warmed hair care compositions can provide enhanced efficacy, i.e., can provide improved benefits. For example, warmed hair shampoo compositions can provide improved cleaning benefits, warmed hair styling compositions can provide improved styling benefits, warmed hair conditioning compositions can provide improved hair conditioning benefits due to improved penetration of ingredients, warmed hair color compositions and warmed hair growth compositions can also provide improved benefits.

### DETAILED DESCRIPTION

While the specification concludes with claims particularly pointing out and distinctly claiming the invention, it is believed the present invention will be better understood from the following description.

All percentages are by weight of the total composition unless otherwise indicated. All ratios are weight ratios unless otherwise indicated. All percentages, ratios, and levels of ingredients referred to herein are based on the actual amount of the ingredient, and do not include solvents, fillers, or other materials with which the ingredient may be combined as commercially available products, unless otherwise indicated.

As used herein, “comprising” means that other steps and other ingredients which do not affect the end result can be added. This term encompasses the terms “consisting of” and “consisting essentially of”.

All cited references are incorporated herein by reference in their entireties. Citation of any reference is not an admission regarding any determination as to its availability as prior art to the claimed invention.

Hair Care Kit

The hair care kits of the present invention are used for warming hair care compositions by a heat from heat generating agents. In the hair care kits of the present invention, preferably, the hair care composition is warmed before applying it to the hair. The hair care compositions are warmed to a temperature of, preferably from about 25° C. to about 80° C., more preferably from about 30° C. to about 60° C. This temperature can be adjusted by, for example, choosing the heat generating agents, the amount of the heat generating agent, additional agents which can control a heat generating reaction, and materials used as the package of the kits.

Kit A

The hair care kit A of the present invention comprises an aqueous hair care composition and a heat generating agent which generates a heat by mixing with the aqueous hair care composition, wherein the heat generating agent is isolated from the aqueous hair care composition.

The heat generating agents generate a heat when the heat generating agents are mixed with the aqueous hair care compositions in kit A. Thus, before applying the aqueous hair care compositions to the hair, the heat generating agents are mixed with the compositions in kit A in order to warm the compositions.

In kit A, the aqueous hair care compositions and the heat generating agents can be packed into the same container, or the aqueous hair care compositions and the heat generating agents can be packed into different containers, i.e., the aqueous hair care compositions can be packed into a first container and the heat generating agents can be packed into a second container.

When the aqueous hair care compositions and the heat generating agents are packed into different containers, the heat generating agents can be mixed with the aqueous hair care composition, for example, by releasing the heat generating agents and the aqueous hair care composition from each container and mixing them together in another container or on hands, or by releasing the heat generating agent from the second container and adding it into the first container, or by releasing the aqueous hair care composition from the first container and adding it into the second container.

When the aqueous hair care compositions and the heat generating agents are packed into the same container, preferably, the container comprises two compartments, and the aqueous hair care compositions and the heat generating agents are packed into different compartments, i.e., the aqueous hair care compositions are packed into a first compartment and the heat generating agents are packed into a second compartment. In the container having two compartments, the first and second compartments are preferably isolated by a breakable partition. In the container having two compartments, the first and second compartments can be isolated with each other by a breakable partition, and the first and second compartments can be positioned so that first or second compartment is encompassed with the other.

When the container having two compartments is used, the heat generating agents can be mixed with the aqueous hair care composition, for example, by releasing the heat generating agents and the aqueous hair care composition from each compartment and mixing them together in another container or on hands. When the container having two compartments isolated by breakable partitions are used, the heat generating agents can be mixed with the aqueous hair care compositions in the same container, for example, by breaking the breakable partitions.

Kit B

The hair care kit B of the present invention comprises:

(i) a hair care composition;

(ii) a heat generating agent which generates a heat by reaction with a reacting means; and

(iii) a reacting means;

wherein the hair care composition, the heat generating agent, and the reacting means are isolated respectively.

The heat generating agents generate a heat by reaction with reacting means in kit B. Thus, before applying the hair care compositions to the hair, the reaction with the reacting means is started, for example, by mixing the reacting means with the heat generating agent in kit B in order to warm the compositions.

In kit B, the hair care compositions, the heat generating agents, and the reacting means can be packed into the same container. In kit B, the hair care compositions, the heat generating agents, and the reacting means can be packed into different containers respectively, i.e., the hair care compositions are packed into a first container, the heat generating agents are packed into a second container, and the reacting means are packed into a third container.

When the hair care compositions, the heat generating agents, and the reacting means are packed into different containers, the heat generating agents can be mixed with the reacting means, for example, by releasing the heat generating agents and the reacting means from each container and mixing them together in another container, or by releasing the heat generating agent from the second container and adding it into the third container, or by releasing the reacting means from the third container and adding it into the second container. When the hair care compositions, the heat generating agents, and the reacting means are packed into different containers, the hair care compositions can be warmed, for example, by contacting the container of the hair care compositions with the container which the heat generating agents and the reacting means are mixed in.

When the hair care compositions, the heat generating agents, and the reacting means are packed into the same container, preferably, the container comprises three compartments, and the hair care compositions, the heat generating agents, and the reacting means are packed into different compartments respectively, i.e., the hair care compositions are packed into a first compartment, the heat generating agents are packed into a second compartment, and the reacting means are packed into a third compartment. In the container having three compartments, the second and third compartments are preferably isolated by a breakable partition. In the container having three compartments, the second and third compartments are preferably isolated with each other by a breakable partition, the second and third compartments can be positioned so that the second or third compartment is encompassed with the other. In the container having three compartments, the first compartment is preferably isolated from the second and third compartments by a non-breakable and heat conductive partition.

When the container having three compartments is used, the heat generating agents can be mixed with the reacting means, for example, by releasing the heat generating agents and the reacting means from each compartment and mixing them together in another container. When the container having three compartments wherein the second and third compartments are isolated by the breakable partitions is used, the generating agents can be mixed with the reacting means in the same container, for example, by breaking the breakable partitions. When the container having three compartments is used, the hair care compositions can be warmed, for example, by contacting the compartment of the hair care compositions with the container which the heat generating agents and the reacting means are mixed in, or the hair care compositions can be warmed through the non-breakable and heat conductive partition.

Heating Device

The heating devices of the present invention are used for warming hair care compositions. The hair care compositions are warmed to a temperature of, preferably from about 25° C. to about 80° C., more preferably from about 30° C. to about 60° C. This temperature can be adjusted by, for example, choosing the heat generating agents, the amount of the heat generating agent, additional agents which can control a heat generating reaction, and materials used as the package of the heat device.

The heating devices of the present invention can be used for warming a hair care composition before applying it to the hair, or can be used for warming a hair care composition after applying it to the hair.

The heating devices of the present invention can be provided in the shape of a container suitable for receiving the hair care composition. The container can be used for warming hair care composition before the hair care composition is applied to the hair. The heating devices of the present invention can be also provided in the shape suitable for covering the hair, for example, the shape of a cap. The cap can be used for warming hair care composition after the hair care composition is applied to the hair.

Heating Source

The heating device of the present invention comprises a heating source. The heating source useful herein includes, the combination of a heat generating agent and a reacting means, heat reserving materials, resistive heating systems, electromagnetic induction heating systems, and mixtures thereof.

The combination of a heat generating agent and a reacting means, wherein the heat generating agent can generate a heat by reaction with the reacting means, can be used as a heating source of the present invention. The reaction with the reacting means can be started by, for example, by mixing the heat generating agent with the reacting means. When the heat generating agent and the reacting means as a heating source, the heat generating agents is isolated from the reacting means. When the heat generating agent and the reacting means are used as a heating source, preferably, the heating source further comprises two compartments, the heat generating agent and the reacting means are packed into different compartments, i.e., the heat generating agents are packed into a first compartment and the reacting means are packed into a second compartment. In the heating source comprising two compartments, the first and second compartments are preferably isolated by a breakable partition. In the heating source comprising two compartments, the first and second compartments are preferably isolated with each other by a breakable partition, the first and second compartments can be positioned so that the first or second compartment is encompassed with the other. In the heating source comprising two compartments isolated by the breakable partitions, the generating agents can be mixed with reacting means in the same container, for example, by breaking the breakable partitions.

The heat reserving materials can be used as a heating source of the present invention. The heat reserving materials useful herein are those which can reserve a heat. The heat reserving materials can reserve a heat, for example, by boiling in a hot water, by microwave, and by electric heating systems. The heat reserving materials include, for example, silica gel, carboxymethyl cellulose gel, phase-changing materials, and mixtures thereof.

The resistive heating materials can be used as a heating source of the present invention. The resistive heating materials include, for example, nichrome wire, ceramics, electrically conductive polymers, and mixtures thereof.

The heating source of the present invention can be covered by a non-breakable and heat conductive layer.

Heat Generating Agent

The hair care kits A and B comprise a heat generating agent. The heat generating agents useful in kit A are those generating a heat by mixing with a aqueous hair care composition contained in kit A. The heat generating agents useful in kit B are those generating a heat by reaction with a reacting means contained in kit B.

The heating devices of the present invention may comprise a heat generating agent. The heating agents useful in the heating devices of the present invention are those generating a heat by reaction with a reacting means contained together with the heat generating agents in the heating devices.

The heat generating agents useful in kits A and B and the heating devices include, for example, the combination of iron, active carbon, and chloride, the combination of iron and potassium peroxodisulfate, calcium oxide, magnesium oxide, magnesium sulfate, calcium chloride, magnesium, magnesium chloride, iron (II) chloride, iron (III) chloride, zeolite, polyhydric alcohol, and mixtures thereof. These agents can generate a heat via a reaction with water. The chloride useful herein, which is used together with the iron and active carbon, includes, for example, sodium chloride, potassium chloride, copper chloride, ferric chloride, ferrous chloride, and mixtures thereof. The agents comprising iron and/or magnesium are preferably mixed with water in the presence of oxygen. The polyhydric alcohol useful herein includes, for example, 1,2-propane diol or propylene glycol, 1,3-propane diol, hexylene glycol, glycerin, diethylene glycol, dipropylene glycol, 1,2-butylene glycol, 1,4-butylene glycol, and mixtures thereof. Preferred polyhydric alcohol useful herein is glycerin.

The heating agents comprising solid materials such as zeolite and magnesium sulfate can be dispersed in an inert carrier, in order to aid mixing with the hair conditioning compositions in kit A and the reacting means in kit B and in the heating devices, and in order to prevent unexpected reactions before use. The inert carrier can be a liquid or a solid such as powders. The inert carrier useful herein includes, for example, powder polyethylene glycol, liquid polyethylene glycol, powder polypropylene glycol, liquid polypropylene glycol, and mixtures thereof. The polyhydric alcohol described above can be also used as inert carriers.

The inert carrier may contain a viscosity modifying agent. The viscosity modifying agent useful herein includes, for example, vinyl polymers such as cross linked acrylic acid polymers with the CTFA name Carbomer, carboxylic acid/carboxylate copolymers such as acrylic acid/alkyl acrylate copolymers with the CTFA name Acrylates/C10-30 Alkyl Acrylate Crosspolymer, cellulose derivatives and modified cellulose polymers, polyvinylpyrrolidone, polyvinyl alcohol, guar gum, other gums, starch-based polymers, alginic acid-based polymers, acrylate polymers, polyalkylene glycols having a molecular weight of more than about 1000, inorganic water soluble material such as bentonite, aluminum magnesium silicate, laponite, hectonite, and anhydrous silicic acid, and mixtures thereof.

The heat generating agents useful in kit B and the heating devices further include, for example, hydrogen peroxide, supercooled liquid, benzoline, and mixtures thereof. Hydrogen peroxide, supercooled liquid, and benzoline can generate a heat via different reactions with different reacting means respectively. Hydrogen peroxide can generate a heat via a reduction reaction with ascorbic acid. Supercooled liquid can generate a heat via phase change from a supercooled liquid to a solid by a stimulating means. The supercooled liquid useful herein includes, for example, sodium acetates such as sodium acetate, sodium acetate monohydrate, sodium acetate dihydrate, sodium acetate trihydrate, and aqueous solutions of these sodium acetates, and mixtures thereof. Preferred supercooled liquid useful herein is sodium acetate trihydrate.

Benzoline can be also used as a heat generating agent in kit B and the heating device. Benzoline generates a heat via conbusting slowly in the presence of a catalyst.

Preferred heat generating agents in kit A are calcium oxide, magnesium oxide, magnesium, magnesium sulfate, zeolite, and mixtures thereof.

Preferred heat generating agents in kit B are calcium oxide, magnesium oxide, magnesium, magnesium sulfate, zeolite, supercooled liquid such as sodium acetate trihydrate, and mixtures thereof.

Preferred heat generating agents in the heating device are calcium oxide, magnesium oxide, magnesium, magnesium sulfate, zeolite, supercooled liquid such as sodium acetate trihydrate, and mixtures thereof.

Reacting Means

The hair care kit B of the present invention comprises a reacting means which is used for reacting the heat generating agent. When the heat generating agent is selected from the group consisting of the combination of iron, active carbon, and chloride, the combination of iron and potassium peroxodisulfate, calcium oxide, magnesium oxide, magnesium sulfate, calcium chloride, magnesium, magnesium chloride, iron (II) chloride, iron (III) chloride, zeolite, polyhydric alcohol, and mixtures thereof, the reacting means useful herein are water, aqueous solutions, or aqueous compositions, wherein the reaction of the heat generating agent with the reacting means is started by mixing the heat generating agent with the reacting means. When hydrogen peroxide is used as a heat generating agent, the reacting means useful herein is ascorbic acid, wherein the reaction of the heat generating agent with the reacting means is started by mixing the heat generating agent with the reacting means. When the supercooled liquid is used as a heat generating agent, the reacting means useful herein are stimulating means which can provide a stimulation to supercooled liquid in order to start the phase change reaction of the supercooled liquid. The stimulating means useful herein include, for example, a stimulating means which can provide a stimulation by mixing with supercooled liquid. Such stimulating means include, for example, crystal forms of material which is used for the supercooled liquid, wherein the reaction of the supercooled liquid is started by mixing the stimulating means with the supercooled liquid. The stimulating means useful herein also include, for example, a stimulating means which provides a physical stimulation such as pressure and vibration. Such stimulating means include, for example, a switch which provides a physical stimulation such as pressure and vibration, wherein the reaction of the supercooled liquid is started by, for example, turning on the switch. When benzoline is used as a heat generating agent, the reacting means useful herein are catalysts such as platinum, wherein the reaction of the heat generating agent with the reacting means is started by mixing the heat generating agent with the reacting means.

Reaction Control Agents

The hair care kits A and B, and the heating devices may further comprise reaction control agents which can control a heat generating reaction of the heat generating agent. The reaction control agents may slow down the reaction, or accelerate the reaction. The reaction control agents may also be able to control a temperature which the hair care composition is warmed to.

When the heat generating agent such as calcium oxide, magnesium, magnesium oxide, and mixtures thereof, is used, the reaction control agents are preferably used for promoting the reaction of the heat generating agent. Acids can be used as reaction control agents for promoting the reaction of the heat generating agent such as calcium oxide, magnesium, magnesium oxide, and mixtures thereof. The acid useful herein includes, for example, citric acid, sodium diphosphate, potassium diphophate. λ-glutamic acid, lactic acid, hydrochloric acid, malic acid, succinic acid, acetic acid, fumaric acid, λ-glutamic acid hydrochloride, tartaric acid, and mixtures thereof; preferably λ-glutamic acid, lactic acid, hydrochloric acid, and mixtures thereof. Among the above acids, citric acid is preferably used herein. The acid is contained at a level such that the mole ratio of the heat generating agent to acid is from about 1:0.1 to about 1:10, preferably from about 1:0.5 to about 1:5. The acid can be isolated from others such as hair care compositions, heat generating agents, and reacting means in the kits and the device. The acid can be incorporated in others such as hair care compositions, heat generating agents, and reacting means in the kits and the device.

When the heat generating agent such as calcium oxide, magnesium, magnesium oxide, and mixtures thereof, is used, the reaction control agents are preferably used for slowing down the reaction of the heat generating agent. Water absorbing polymer can be used as reaction control agents for slowing down the reaction of the heat generating agent such as calcium oxide, magnesium, magnesium oxide, and mixtures thereof. The water absorbing polymer useful herein includes, for example, vinyl polymers such as cross linked acrylic acid polymers with the CTFA name Carbomer, carboxylic acid/carboxylate copolymers such as acrylic acid/alkyl acrylate copolymers with the CTFA name Acrylates/C10-30 Alkyl Acrylate Crosspolymer, cellulose derivatives and modified cellulose polymers such as Hydroxyethylcellulose, polyvinylpyrrolidone, polyvinyl alcohol, guar gum, other gums, starch-based polymers, alginic acid-based polymers, acrylate polymers, polyalkylene glycols having a molecular weight of more than about 1000, and mixtures thereof. Among the above water absorbing polymers, Hydroxyethylcellulose is preferably used herein. The water absorbing polymers can be isolated from others such as hair care compositions, heat generating agents, and reacting means in the kits and the device. The water absorbing polymers can be incorporated in others such as hair care compositions, heat generating agents, and reacting means in the kits and the device.

Heat Reserving Materials

The hair care kits A and B, and the heating devices may further comprise heat reserving materials which can reserve a heat. The heat reserving materials include, for example, silica gel, carboxymethyl cellulose gel, phase-changing materials, and mixtures thereof. The phase-changing materials useful herein are those which have a melting point of from about 25° C. to about 80° C. The phase-changing materials useful herein includes, for example, a fatty compound such as fatty alcohol and fatty acid; hydrocarbons; a mixture of hydrocarbons and foamed polyolefin; and mixtures thereof. Fatty compound useful herein are disclosed below under the title “HIGH MELTING POINT FATTY COMPOUND”.

In the kits of the present invention, the heat reserving material can be used for prolonging heating. In the heating device of the present invention, the heat reserving materials can be used alone as heating source, or can be used in combination with others such as resistive heating system and the combination of a heat generating agent and a reacting means.

The heat reserving materials can be isolated from others such as hair care compositions, heat generating agents, and reacting means in the kits and the device. For example, in kit A, the heat reserving materials may be positioned close to both of hair care compositions and heat generating agents. For example, in kit B, the heat reserving materials may be positioned between hair care compositions and the combination of heat generating agents and reacting means. For example, the heat reserving materials may be positioned surrounding other heating sources.

The heat reserving materials can be incorporated in others such as hair care compositions, heat generating agents, and reacting means in the kits and the device. For example, in kit A, the heat reserving materials can be incorporated in hair care compositions, or heat generating agents. For example, in kit B, the heat reserving materials can be incorporated in hair care compositions, heat generating agents, or reacting means. For example, in the heating device, the heat reserving materials can be incorporated in heat generating agents, or reacting means.

Hair Care Composition

The hair care kits and the heating devices of the present invention are used for warming hair care compositions, preferably for warming hair conditioning compositions. Various hair care compositions can be used in the present invention. In kit A, aqueous hair care compositions are used. In kit B and the heating devices, both aqueous and non-aqueous hair care compositions can be used. The aqueous/non-aqueous hair care compositions useful herein include, aqueous/non-aqueous hair shampoo compositions, aqueous/non-aqueous hair styling compositions, aqueous/non-aqueous hair conditioning compositions, aqueous/non-aqueous hair color compositions, aqueous/non-aqueous hair growth compositions, and mixtures thereof.

It is believed that; warmed hair care compositions can provide enhanced benefits, for example, warmed hair shampoo compositions can provide improved cleaning benefits, warmed hair styling compositions can provide improved styling benefits, warmed hair conditioning compositions can provide improved hair conditioning benefits due to improved penetration of ingredients, warmed hair color compositions and warmed hair growth compositions can also provide improved benefits.

The hair care compositions of the present invention can be in the form of rinse-off products or leave-on products, can be transparent or opaque, and can be formulated in a wide variety of product forms, including but not limited to creams, gels, emulsions, mousses, and sprays.

Hair Conditioning Composition

The heating devices of the present invention are preferably used for warming a hair conditioning composition. The hair care kits of the present invention preferably comprises a hair conditioning composition. In kit A, aqueous hair conditioning compositions are used. In kit B, both aqueous and non-aqueous hair conditioning compositions can be used. The hair conditioning composition useful herein includes a composition (hereinafter “Composition A”) comprising by weight:

(a) from about 0.1% to about 15% of a high melting point fatty compound;

(b) from about 0.1% to about 10% of an amidoamine having the following general formula:

R¹ CONH (CH₂)ₘ N (R²)₂

wherein R¹ is a residue of C₁₁ to C₂₄ fatty acids, R² is a C₁ to C₄ alkyl, and m is an integer from 1 to 4;

(c) an acid selected from the group consisting of λ-glutamic acid, lactic acid, hydrochloric acid, malic acid, succinic acid, acetic acid, fumaric acid, λ-glutamic acid hydrochloride, tartaric acid, and mixtures thereof, at a level such that the mole ratio of amidoamine to acid is from about 1:0.3 to about 1:1; and

(d) an aqueous carrier, and a composition (hereinafter “Composition B”) comprising by weight:

(a) from about 0.1% to about 15% of a high melting point fatty compound having a melting point of 25° C. or higher;

(b) from about 0.1% to about 10% of a cationic conditioning agent;

(c) from about 0.1% to about 10% of a low melting point oil having a melting point of less than 25° C.; and

(d) an aqueous carrier.

High Melting Point Fatty Compound

The hair conditioning compositions A and B of the present invention comprise a high melting point fatty compound. The high melting fatty compound, together with a cationic surfactant such as an amidoamine and an aqueous carrier, provide a gel network which is suitable for providing various conditioning benefits such as slippery and slick feel on wet hair, and softness, moisturized feel, and fly-away control on dry hair.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The high melting point fatty compound is included in the compositions A and B at a level by weight of from about 0.1% to about 15%, preferably from about 0.25% to about 13%. More preferably, the high melting point fatty compound is included at a level by weight of from about 1 % to about 10% especially in the composition A, at a level by weight of from about 0.25% to about 5% especially in the composition B.

The fatty alcohols useful herein are those having from about 14 to about 30 carbon atoms, preferably from about 16 to about 22 carbon atoms. These fatty alcohols are saturated and can be straight or branched chain alcohols. Nonlimiting examples of fatty alcohols include, cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-1 0, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

Commercially available high melting point fatty compounds useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago Ill., USA), HYSTRENE available from Witco Corp. (Dublin Ohio, USA), and DERMA available from Vevy (Genova, Italy).

Amidoamine

The hair conditioning composition A of the present invention comprises an amidoamine of the following general formula:

R¹ CONH (CH₂)ₘ N (R²)₂

wherein R¹ is a residue of C₁₁ to C₂₄ fatty acids, R² is a C₁ to C₄ alkyl, and m is an integer from 1 to 4.

The amidoamine is included in the composition A at a level by weight of from about 0.1% to about 10%, preferably from about 0.25% to about 8%, more preferably from about 0.5% to about 3%.

The amidoamine can be also included in the composition B as a cationic conditioning agent at a level by weight of, preferably from about 0.1% to about 10%, more preferably from about 0.25% to about 8%, still more preferably from about 0.5% to about 3%.

Preferred amidoamines useful in the present invention includes stearamidopropyidimethylamine, stearamidopropyidiethylamine, stearamidoethyldiethylamine, stearamidoethyidimethylamine, palmitamidopropyidimethylamine, palmitamidopropyidiethylamine, palmitamidoethyldiethylamine, palmitamidoethyldimethylamine, behenamidopropyldimethylamine, behenamidopropyldiethylamine, behenamidoethyidiethylamine, behenamidoethyldimethylamine, arachidamidopropyldimethylamine, arachidamidopropyldiethylamine, arachidamidoethyldiethylamine, arachidamidoethyldimethylamine, and mixtures thereof; more preferably stearamidopropyidimethylamine, stearamidoethyldiethylamine, and mixtures thereof.

Commercially available amidoamines useful herein include: stearamidopropyldimethylamine having tradename SAPDMA available from Inolex, and tradename Amidoamine MPS available from Nikko.

Acids

The hair conditioning composition A of the present invention comprises an acid selected from the group consisting of λ-glutamic acid, lactic acid, hydrochloric acid, malic acid, succinic acid, acetic acid, fumaric acid, λ-glutamic acid hydrochloride, tartaric acid, and mixtures thereof; preferably λ-glutamic acid, lactic acid, hydrochloric acid, and mixtures thereof. The acid is contained at a level such that the mole ratio of amidoamine to acid is from about 1:0.3 to about 1:1, preferably from about 1:0.5 to about 1:0.9.

The acid can be also included in the composition B at a level such that the mole ratio of amidoamine to acid is from about 1:0.3 to about 1:1, preferably from about 1:0.5 to about 1:0.9.

Commercially available acids useful herein include: λ-Glutamic acid: λ-Glutamic acid (cosmetic grade) available from Ajinomoto.

Particles

The compositions A and B of the present invention comprise a particle. The particle is included in the compositions A and B at a level by weight of from about 0.01% to about 10%, preferably from about 0.1% to about 5%, more preferably from about 0.1% to about 2%. The particles useful herein has an average particle size of preferably from about 25 μm to about 1500 μm, more preferably from about 50 μm to about 1000 μm, still more preferably from about 50 μm to about 500 μm. Both organic and inorganic particles can be used herein. Preferred particles useful herein include organic particles such as cellulose particles, and inorganic particles such as mica, silica, mud, clay, zeolite and mixtures thereof. More preferred is silica. Preferred particles useful herein can be those having a breakability such that the particles are breakable when the particles contained in the compositions are spread on the hands and/or on the hair.

Commercially available particles useful herein include: silica having tradename Neosil series such as Neosil CBT 60 available from Crosfield.

Aqueous Carrier

The compositions A and B of the present invention comprise an aqueous carrier. The level and species of the carrier are selected according to the compatibility with other components, and other desired characteristic of the product.

The carrier useful in the present invention include water and water solutions of lower alkyl alcohols and polyhydric alcohols. The lower alkyl alcohol useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 95%, preferably from about 30% to about 92%, and more preferably from about 50% to about 90% water.

Cationic Conditioning Agent

The hair conditioning composition B of the present invention comprises a cationic conditioning agent. This cationic conditioning agent, together with the high melting point fatty compounds, provide a gel network suitable for providing various conditioning benefits such as slippery and slick feel on wet hair, and such as softness, moisturized feel, and fly-away control on dry hair.

The cationic conditioning agent is included in the composition at a level by weight of from about 0.1% to about 10%, preferably from about 0.25% to about 8%, more preferably from about 0.5% to about 3%.

The cationic conditioning agent can be also included in the composition A at a level by weight of, preferably from about 0.1% to about 10%, more preferably from about 0.25% to about 8%, still more preferably from about 0.5% to about 3%.

The cationic conditioning agent is selected from the group consisting of cationic surfactants, cationic polymers, and mixtures thereof.

Cationic Surfactant

The cationic surfactant useful herein is any known to the artisan, and can be included in the composition at a level by weight of, preferably from about 0.1 % to about 10%, more preferably from about 0.25% to about 8%, still more preferably from about 0.5 to about 3%.

Among the cationic surfactants useful herein are those corresponding to the general formula (I):

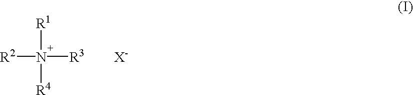

wherein at least one of R¹, R², R³, and R⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R¹, R², R³, and R⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R¹, R², R³, and R⁴ are independently selected from C₁ to about C₂₂ alkyl. Nonlimiting examples of cationic surfactants useful in the present invention include the materials having the following CTFA designations: quaternium-8, quaternium-14, quaternium-18, quaternium-18 methosulfate, quaternium-24, and mixtures thereof.

Among the cationic surfactants of general formula (I), preferred are those containing in the molecule at least one alkyl chain having at least 16 carbons. Nonlimiting examples of such preferred cationic surfactants include: behenyl trimethyl ammonium chloride available, for example, with tradename INCROQUAT TMC-80 from Croda and ECONOL TM22 from Sanyo Kasei; cetyl trimethyl ammonium chloride available, for example, with tradename CA-2350 from Nikko Chemicals, hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride.

Also preferred are hydrophilically substituted cationic surfactants in which at least one of the substituents contain one or more aromatic, ether, ester, amido, or amino moieties present as substituents or as linkages in the radical chain, wherein at least one of the R¹-R⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Preferred hydrophilically substituted cationic surfactants include those of the formula (II) through (VIII) below:

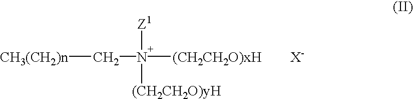

wherein n is from 8 to about 28, x+y is from 2 to about 40, Z¹ is a short chain alkyl, preferably a C₁-C₃ alkyl, more preferably methyl, or (CH₂CH₂O)_{z}H wherein x+y+z is up to 60, and X is a salt forming anion as defined above;

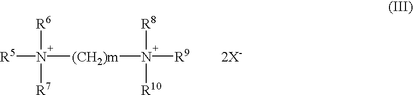

wherein m is 1 to 5, one or more of R⁵, R⁶, and R⁷ are independently an C₁ - C₃₀ alkyl, the remainder are CH₂CH₂OH, one or two of R⁸, R⁹, and R¹⁰ are independently an C₁-C₃₀ alkyl, and remainder are CH₂CH₂OH, and X is a salt forming anion as mentioned above;

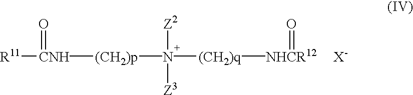

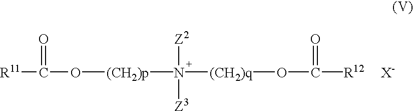

wherein, independently for formulae (IV) and (V), Z² is an alkyl, preferably a C₁-C₃ alkyl, more preferably methyl, and Z³ is a short chain hydroxyalkyl, preferably hydroxymethyl or hydroxyethyl, p and q independently are integers from 2 to 4, inclusive, preferably from 2 to 3, inclusive, more preferably 2, R¹¹ and R¹² , independently, are substituted or unsubstituted hydrocarbyls, preferably C₁₂-C₂₀ alkyl or alkenyl, and X is a salt forming anion as defined above;

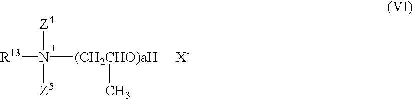

wherein R¹³ is a hydrocarbyl, preferably a C₁-C₃ alkyl, more preferably methyl, Z⁴ and Z⁵ are, independently, short chain hydrocarbyls, preferably C₂-C₄ alkyl or alkenyl, more preferably ethyl, a is from 2 to about 40, preferably from about 7 to about 30, and X is a salt forming anion as defined above;

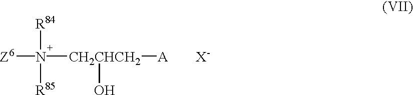

wherein R⁸⁴ and R⁸⁵, independently, are C₁-C₃ alkyl, preferably methyl, Z⁶ is a C₁₂-C₂₂ hydrocarbyl, alkyl carboxy or alkylamido, and A is a protein, preferably a collagen, keratin, milk protein, silk, soy protein, wheat protein, or hydrolyzed forms thereof; and X is a salt forming anion as defined above;

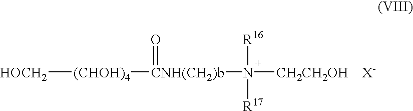

wherein b is 2 or 3, R¹⁶ and R¹⁷, independently are C₁-C₃ hydrocarbyls preferably methyl, and X is a salt forming anion as defined above. Nonlimiting examples of hydrophilically substituted cationic surfactants useful in the present invention include the materials having the following CTFA designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and mixtures thereof.

Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commerically available under the following tradenames; VARISOFT 110, VARIQUAT K1215 and 638 from Witco Chemical, MACKPRO KLP, MACKPRO WLW, MACKPRO MLP, MACKPRO NSP, MACKPRO NLW, MACKPRO WWP, MACKPRO NLP, MACKPRO SLP from Mcintyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas.

Salts of primary, secondary, and tertiary fatty amines are also suitable cationic surfactants. The alkyl groups of such amines preferably have from about 12 to about 22 carbon atoms, and can be substituted or unsubstituted. Particularly useful are salts of amidoamines which are selected from the species disclosed above under the title “AMIDE AMINE” and “ACID”. Preferably, the salts of amidoamines are used as cationic conditioning agents in the composition B.

Cationic Polymer

The cationic polymer useful herein is described below. As used herein, the term “polymer” shall include materials whether made by polymerization of one type of monomer or made by two (i.e., copolymers) or more types of monomers.

Preferably, the cationic polymer is a water-soluble cationic polymer. By “water soluble” cationic polymer, what is meant is a polymer which is sufficiently soluble in water to form a substantially clear solution to the naked eye at a concentration of 0.1% in water (distilled or equivalent) at 25° C. The preferred polymer will be sufficiently soluble to form a substantially clear solution at 0.5% concentration, more preferably at 1.0% concentration.

The cationic polymers hereof will generally have a weight average molecular weight which is at least about 5,000, typically at least about 10,000, and is less than about 10 million. Preferably, the molecular weight is from about 100,000 to about 2 million. The cationic polymers will generally have cationic nitrogen-containing moieties such as quaternary ammonium or cationic amino moieties, and mixtures thereof.

The cationic charge density is preferably at least about 0.1 meq/gram, more preferably at least about 1.5 meq/gram, even more preferably at least about 1.1 meq/gram, still more preferably at least about 1.2 meq/gram. Cationic charge density of the cationic polymer can be determined according to the Kjeldahl Method. Those skilled in the art will recognize that the charge density of amino-containing polymers may vary depending upon pH and the isoelectric point of the amino groups. The charge density should be within the above limits at the pH of intended use.

Any anionic counterions can be utilized for the cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

The cationic nitrogen-containing moiety will be present generally as a substituent, on a fraction of the total monomer units of the cationic hair conditioning polymers. Thus, the cationic polymer can comprise copolymers, terpolymers, etc. of quaternary ammonium or cationic amine-substituted monomer units and other non-cationic units referred to herein as spacer monomer units. Such polymers are known in the art, and a variety can be found in the CTFA Cosmetic Ingredient Dictionary, 3rd edition, edited by Estrin, Crosley, and Haynes, (The Cosmetic, Toiletry, and Fragrance Association, Inc., Washington, D.C., 1982).

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Other suitable spacer monomers include vinyl esters, vinyl alcohol (made by hydrolysis of polyvinyl acetate), maleic anhydride, propylene glycol, and ethylene glycol.

The cationic amines can be primary, secondary, or tertiary amines, depending upon the particular species and the pH of the composition. In general, secondary and tertiary amines, especially tertiary amines, are preferred.

Amine-substituted vinyl monomers can be polymerized in the amine form, and then optionally can be converted to ammonium by a quaternization reaction. Amines can also be similarly quaternized subsequent to formation of the polymer. For example, tertiary amine functionalities can be quaternized by reaction with a salt of the formula R′X wherein R′ is a short chain alkyl, preferably a C₁-C₇ alkyl, more preferably a C₁-C₃ alkyl, and X is an anion which forms a water soluble salt with the quaternized ammonium.

Suitable cationic amino and quaternary ammonium monomers include, for example, vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C₁-C₃ alkyls, more preferably C₁ and C₂ alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C₁- C₇ hydrocarbyls, more preferably C₁-C₃, alkyls.

The cationic polymers hereof can comprise mixtures of monomer units derived from amine- and/or quaternary ammonium-substituted monomer and/or compatible spacer monomers.

Suitable cationic hair conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-1 6), such as those commercially available from BASF Wyandotte Corp. (Parsippany, N.J., USA) under the LUVIQUAT tradename (e.g., LUVIQUAT FC 370); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyidiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and co-polymers of unsaturated carboxylic acids having from 3 to 5 carbon atoms, as described in U.S. Pat. No. 4,009,256, incorporated herein by reference.

Other cationic polymers that can be used include polysaccharide polymers, such as cationic cellulose derivatives and cationic starch derivatives.

Cationic polysaccharide polymer materials suitable for use herein include those of the formula:

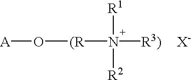

wherein: A is an anhydroglucose residual group, such as a starch or cellulose anhydroglucose residual, R is an alkylene oxyalkylene, polyoxyalkylene, or hydroxyalkylene group, or combination thereof, R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less, and X is an anionic counterion, as previously described.

Cationic cellulose is available from Amerchol Corp. (Edison, N.J., USA) in their Polymer JR® and LR® series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200®.

Other cationic polymers that can be used include cationic guar gum derivatives, such as guar hydroxypropyltrimonium chloride (commercially available from Celanese Corp. in their Jaguar R series). Other materials include quaternary nitrogen-containing cellulose ethers (e.g., as described in U.S. Pat. No. 3,962,418, incorporated herein by reference), and copolymers of etherified cellulose and starch (e.g., as described in U.S. Pat. No. 3,958,581, incorporated herein by reference.)

Low Melting Point Oil

The hair conditioning composition B of the present invention comprises a low melting point oil, which has a melting point of less than 25° C., and is included in the composition at a level by weight of from about 0.1% to about 10%, preferably from about 0.25% to about 6%.

The low melting point oil having a melting point of less than 25° C., can be also included in the composition A at a level by weight of, preferably from about 0.1% to about 10%, more preferably from about 0.25% to about 6%, still more preferably from about 0.3% to about 3%.

The low melting point oil useful herein is selected from the group consisting of hydrocarbon having from 10 to about 40 carbon atoms, unsaturated fatty alcohols having from about 10 to about 30 carbon atoms, unsaturated fatty acids having from about 10 to about 30 carbon atoms, fatty acid derivatives, fatty alcohol derivatives, ester oils, poly α-olefin oils, and mixtures thereof.

Fatty alcohols useful herein include those having from about 10 to about 30 carbon atoms, preferably from about 12 to about 22 carbon atoms, and more preferably from about 16 to about 22 carbon atoms. These fatty alcohols are unsaturated and can be straight or branched chain alcohols. Suitable fatty alcohols include, for example, oleyl alcohol, isostearyl alcohol, tridecylalcohol, decyl tetradecyl alcohol, and octyl dodecyl alcohol. These alcohols are available, for example, from Shinnihon Rika.

Low melting point oils useful herein include pentaerythritol ester oils, trimethylol ester oils, poly α-olefin oils, citrate ester oils, glyceryl ester oils, and mixtures thereof, and the ester oil useful herein is water-insoluble. As used herein, the term “water-insoluble” means the compound is substantially not soluble in water at 25° C.; when the compound is mixed with water at a concentration by weight of above 1.0%, preferably at above 0.5%, the compound is temporarily dispersed to form an unstable colloid in water, then is quickly separated from water into two phases.

Pentaerythritol ester oils useful herein are those having the following formula:

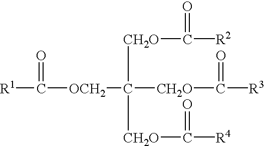

wherein R¹, R², R³, and R⁴, independently, are branched, straight, saturated, or unsaturated alkyl, aryl, and alkylaryl groups having from 1 to about 30 carbons. Preferably, R¹, R², R³, and R⁴, independently, are branched, straight, saturated, or unsaturated alkyl groups having from about 8 to about 22 carbons. More preferably, R¹, R², R³ and R⁴ are defined so that the molecular weight of the compound is from about 800 to about 1200.

Trimethylol ester oils useful herein are those having the following formula:

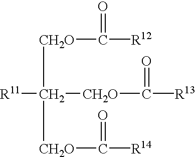

wherein R¹¹ is an alkyl group having from 1 to about 30 carbons, and R¹², R¹³, and R¹⁴, independently, are branched, straight, saturated, or unsaturated alkyl, aryl, and alkylaryl groups having from 1 to about 30 carbons. Preferably, R¹¹ is ethyl and R¹², R¹³, and R¹⁴, independently, are branched, straight, saturated, or unsaturated alkyl groups having from 8 to about 22 carbons. More preferably, R¹¹, R¹², R¹³ and R¹⁴ are defined so that the molecular weight of the compound is from about 800 to about 1200.

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Poly α-olefin oils useful herein are those derived from 1-alkene monomers having from about 6 to about 16 carbons, preferably from about 6 to about 12 carbons atoms. Nonlimiting examples of 1-alkene monomers useful for preparing the poly α-olefin oils include 1-hexene, 1-octene, 1-decene, 1-dodecene, 1-tetradecene, 1-hexadecene, branched isomers such as 4-methyl-1-pentene, and mixtures thereof. Preferred 1-alkene monomers useful for preparing the poly α-olefin oils are 1-octene, 1-decene, 1-dodecene, 1-tetradecene, 1-hexadecene, and mixtures thereof. Poly α-olefin oils useful herein further have a viscosity of from about 1 to about 35,000 cst, a molecular weight of from about 200 to about 60,000, and a polydispersity of no more than about 3.

Poly α-olefin oils having a molecular weight of at least about 800 are useful herein. Such high molecular weight poly α-olefin oils are believed to provide long lasting moisturized feel to the hair. Poly α-olefin oils having a molecular weight of less than about 800 are useful herein. Such low molecular weight poly α-olefin oils are believed to provide a smooth, light, clean feel to the hair.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Mobil Chemical Co.

Citrate ester oils useful herein are those having a molecular weight of at least about 500 having the following formula:

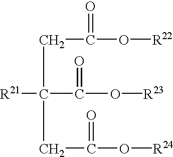

wherein R²¹ is OH or CH₃COO, and R²², R²³, and R²⁴, independently, are branched, straight, saturated, or unsaturated alkyl, aryl, and alkylaryl groups having from 1 to about 30 carbons. Preferably, R²¹ is OH, and R²², R²³, and R²⁴, independently, are branched, straight, saturated, or unsaturated alkyl, aryl, and alkylaryl groups having from 8 to about 22 carbons. More preferably, R²¹, R²², R²³ and R²⁴ are defined so that the molecular weight of the compound is at least about 800.

Particularly useful citrate ester oils herein include triisocetyl citrate with tradename CITMOL 316 available from Bernel, triisostearyl citrate with tradename PELEMOL TISC available from Phoenix, and trioctyidodecyl citrate with tradename CITMOL 320 available from Bernel.

Glyceryl ester oils useful herein are those having a molecular weight of at least about 500 and having the following formula:

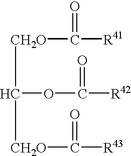

wherein R⁴¹, R⁴², and R⁴³, independently, are branched, straight, saturated, or unsaturated alkyl, aryl, and alkylaryl groups having from 1 to about 30 carbons.

Preferably, R⁴¹, R⁴², and R⁴³, independently, are branched, straight, saturated, or 25 unsaturated alkyl, aryl, and alkylaryl groups having from 8 to about 22 carbons. More preferably, R⁴¹, R⁴², and R⁴³ are defined so that the molecular weight of the compound is at least about 800.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Silicone Compounds

Preferably, the composition A of the present invention may further comprises silicone compound. The silicone compound can be included in the composition A at a level by weight of, preferably from about 0.1% to about 10%, more preferably from about 0.25% to about 8%, still more preferably from about 0.5% to about 3%.

The silicone compound can be also included in the composition B at a level by weight of, preferably from about 0.1% to about 10%, more preferably from about 0.25% to about 8%, still more preferably from about 0.5% to about 3%.

The silicone compounds hereof can include volatile soluble or insoluble, or nonvolatile soluble or insoluble silicone conditioning agents. By soluble what is meant is that the silicone compound is miscible with the carrier of the composition so as to form part of the same phase. By insoluble what is meant is that the silicone forms a separate, discontinuous phase from the carrier, such as in the form of an emulsion or a suspension of droplets of the silicone. The silicone compounds herein may be made by conventional polymerization, or emulsion polymerization.

The silicone compounds for use herein will preferably have a viscosity of from about 1,000 to about 2,000,000 centistokes at 25° C., more preferably from about 10,000 to about 1,800,000, and even more preferably from about 25,000 to about 1,500,000. The viscosity can be measured by means of a glass capillary viscometer as set forth in Dow Corning Corporate Test Method CTM0004, Jul. 20, 1970, which is incorporated by reference herein in its entirety. Silicone compound of high molecular weight may be made by emulsion polymerization.

Silicone compounds useful herein include polyalkyl polyaryl siloxanes, polyalkyleneoxide-modified siloxanes, silicone resins, amino-substituted siloxanes, and mixtures thereof. The silicone compound is preferably selected from the group consisting of polyalkyl polyaryl siloxanes, polyalkyleneoxide-modified siloxanes, silicone resins, and mixtures thereof, and more preferably from one or more polyalkyl polyaryl siloxanes.

Polyalkyl polyaryl siloxanes useful here in include those with the following structure (I)

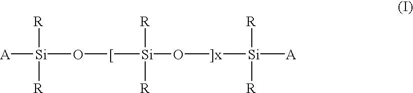

wherein R is alkyl or aryl, and x is an integer from about 7 to about 8,000. “A” represents groups which block the ends of the silicone chains. The alkyl or aryl groups substituted on the siloxane chain (R) or at the ends of the siloxane chains (A) can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable A groups include hydroxy, methyl, methoxy, ethoxy, propoxy, and aryloxy. The two R groups on the silicon atom may represent the same group or different groups. Preferably, the two R groups represent the same group. Suitable R groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their ViscasilR and SF 96 series, and from Dow Corning in their Dow Corning 200 series. Polymethylphenylsiloxanes, for example, from the General Electric Company as SF 1075 methyl phenyl fluid or from Dow Corning as 556 Cosmetic Grade Fluid, are useful herein.

Also preferred, for enhancing the shine characteristics of hair, are highly arylated silicone compounds, such as highly phenylated polyethyl silicone having refractive index of about 1.46 or higher, especially about 1.52 or higher. When these high refractive index silicone compounds are used, they should be mixed with a spreading agent, such as a surfactant or a silicone resin, as described below to decrease the surface tension and enhance the film forming ability of the material.

Another polyalkyl polyaryl siloxane that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. Silicone gums are described by Petrarch, and others including U.S. Pat. No. 4,152,416, to Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. All of these described references are incorporated herein by reference in their entirety. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof.

Polyalkyleneoxide-modified siloxanes useful herein include, for example, polypropylene oxide modified and polyethylene oxide modified polydimethylsiloxane. The ethylene oxide and polypropylene oxide level should be sufficiently low so as not to interfere with the dispersibility characteristics of the silicone. These material are also known as dimethicone copolyols.

Silicone resins, which are highly crosslinked polymeric siloxane systems, are useful herein. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art. Without being bound by theory, it is believed that the silicone resins can enhance deposition of other silicone compounds on the hair and can enhance the glossiness of hair with high refractive index volumes.

Other useful silicone resins are silicone resin powders such as the material given the CTFA designation polymethylsilsequioxane, which is commercially available as Tospearl™ from Toshiba Silicones.

Silicone resins can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the mono-functional unit (CH₃)₃SiO)_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein -which are preferred are MO, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

Amino-substituted siloxanes useful herein include those represented by the following structure (II)

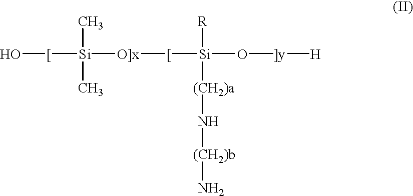

wherein R is CH₃ or OH, x and y are integers which depend on the molecular weight, the average molecular weight being approximately between 5,000 and 10,000. This polymer is also known as “amodimethicone”.

Suitable amino-substituted siloxane fluids include those represented by the formula (III)

(R₁)ₐG₃₋ₐ—Si—(—OSiG₂)ₙ-(—OSiG_{b}(R₁)_{2-b)m}—O—SiG₃₋ₐ(R₁)ₐ (III)

in which G is chosen from the group consisting of hydrogen, phenyl, OH, C₁-C₈ alkyl and preferably methyl; a denotes 0 or an integer from 1 to 3, and preferably equals 0; b denotes 0 or 1 and preferably equals 1; the sum n+m is a number from 1 to 2,000 and preferably from 50 to 150, n being able to denote a number from 0 to 1,999 and preferably from 49 to 149 and m being able to denote an integer from 1 to 2,000 and preferably from 1 to 10; R₁ is a monovalent radical of formula CqH_{2q}L in which q is an integer from 2 to 8 and L is chosen from the groups

—N(R₂)CH₂—CH₂—N(R₂)₂

—N(R₂)₂

—N(R₂)₃A⁻

—N(R₂)CH₂—CH₂—NR₂H₂A³¹

in which R₂ is chosen from the group consisting of hydrogen, phenyl, benzyl, a saturated hydrocarbon radical, preferably an alkyl radical containing from 1 to 20 carbon atoms, and A⁻ denotes a halide ion.

An especially preferred amino-substituted siloxane corresponding to formula (III) is the polymer known as “trimethylsilylamodimethicone”, of formula (IV):

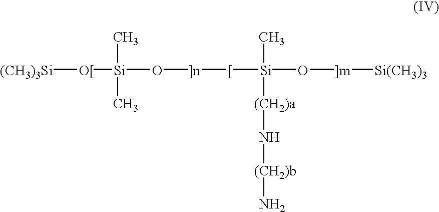

In this formula n and m are selected depending on the molecular weight of the compound desired.

Other amino-substituted siloxane which can be used are represented by the formula (V):

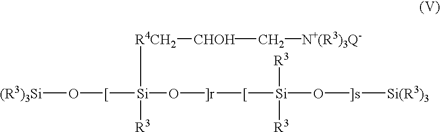

where R³ denotes a monovalent hydrocarbon radical having from 1 to 18 carbon atoms, preferably an alkyl or alkenyl radical such as methyl; R₄ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; r denotes an average statistical value from 2 to 20, preferably from 2 to 8; s denotes an average statistical value from 20 to 200, and preferably from 20 to 50. A preferred polymer of this class is available from Union Carbide under the name “UCAR SILICONE ALE 56.”

Polypropylene Glycol

Preferably, the composition A of the present invention may further comprises a polypropylene glycol. The polypropylene glycol can be included in the composition A at a level by weight of, preferably from about 0.1% to about 10%, more preferably from about 0.25% to about 6%.

The polypropylene glycol can be also included in the composition B at a level by weight of, preferably from about 0.1% to about 10%, more preferably from about 0.25% to about 6%.

The polypropylene glycol useful herein may has a weight average molecular weight of preferably from about 200 g/mol to about 100,000 g/mol, more preferably from about 1,000 g/mol to about 60,000 g/mol. Without intending to be limited by theory, it is believed that the polypropylene glycol herein deposits onto, or is absorbed into hair to act as a moisturizer buffer, and/or provides one or more other desirable hair conditioning benefits. As used herein, the term “polypropylene glycol” includes single-polypropylene glycol-chain segment polymers, and multi-polypropylene glycol-chain segment polymers. The general structure of branched polymers such as the multi-polypropylene glycol-chain segment polymers herein are described, for example, in “Principles of Polymerization,” pp. 17-19, G. Odian, (John Wiley & Sons, Inc., 3^{rd} ed., 1991).

The polypropylene glycol herein are typically polydisperse polymers. The polypropylene glycols useful herein have a polydispersity of from about 1 to about 2.5, preferably from about 1 to about 2, and more preferably from about 1 to about 1.5. As used herein, the term “polydispersity” indicates the degree of the molecular weight distribution of the polymer sample. Specifically, the polydispersity is a ratio, greater than 1, equal to the weight average molecular weight divided by the number average molecular weight. For a further discussion about polydispersity, see “Principles of Polymerization,” pp. 20-24, G. Odian, (John Wiley & Sons, Inc., ₃^{rd} ed., 1991).

The polypropylene glycol useful herein may be either water-soluble, water-insoluble, or may have a limited solubility in water, depending upon the degree of polymerization and whether other moieties are attached thereto. The desired solubility of the polypropylene glycol in water will depend in large part upon the form (e.g., leave-on, or rinse-off form) of the hair care composition. The solubility in water of the polypropylene glycol herein may be chosen by the artisan according to a variety of factors. Accordingly, for a leave-on hair care composition, it is preferred that the polypropylene glycol herein be a water-soluble polypropylene glycol. Solubility information is readily available from polypropylene glycol suppliers, such as Sanyo Kasei (Osaka, Japan). However, the present invention may also take the form of a rinse-off hair care composition. Without intending to be limited by theory, it is believed that in such a composition, a water-soluble polypropylene glycol may be too easily washed away before it effectively deposits on hair and provides the desired benefit(s). For such a composition, a less soluble, or even a water-insoluble polypropylene glycol is therefore preferred. Accordingly, for a rinse-off hair care composition, it is preferred that the polypropylene glycol herein has a solubility in water at 25° C. of less than about 1 g/100 g water, more preferably a solubility in water of less than about 0.5 g/100 g water, and even more preferably a solubility in water of less than about 0.1 g/100 g water.

Preferably the polypropylene glycol is selected from the group consisting of a single-polypropylene glycol-chain segment polymer, a multi-polypropylene glycol-chain segment polymer, and mixtures thereof, more preferably selected from the group consisting of a single-polypropylene glycol-chain segment polymer of Formula I, below, a multi-polypropylene glycol-chain segment polymer of Formula II, below, and mixtures thereof.

Single-Polypropylene Glycol-Chain Segment Polymer

Accordingly, a highly preferred single-polypropylene glycol-chain segment polymer has the formula:

HO—(C₃H₆O)ₐH (III),

wherein a is a value from about 4 to about 400, preferably from about 20 to about 100, and more preferably from about 20 to about 40.

The single-polypropylene glycol-chain segment polymer useful herein is typically inexpensive, and is readily available from, for example, Sanyo Kasei (Osaka, Japan), Dow Chemicals (Midland, Mich., USA), Calgon Chemical, Inc. (Skokie, Ill., USA), Arco Chemical Co. (Newton Square Pa., USA), Witco Chemicals Corp. (Greenwich, Conn., USA), and PPG Specialty Chemicals (Gurnee, Ill., USA).

Multi-Polypropylene Glycol-Chain Segment Polymer

A highly preferred multi-polypropylene glycol-chain segment polymer has the formula:

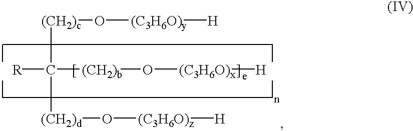

wherein n is a value from about 0 to about 10, preferably from about 0 to about 7, and more preferably from about 1 to about 4. In Formula IV, each R″ is independently selected from the group consisting of H, and C₁-C₃₀ alkyl, and preferably each R″ is independently selected from the group consisting of H, and C₁-C₄ alkyl. In Formula IV, each b is independently a value from about 0 to about 2, preferably from about 0 to about 1, and more preferably b=0. Similarly, c and d are independently a value from about 0 to about 2, preferably from about 0 to about 1. However, the total of b+c+d is at least about 2, preferably the total of b+c+d is from about 2 to about 3. Each e is independently a value of 0 or 1, if n is from about 1 to about 4, then e is preferably equal to 1. Also in Formula IV, x, y, and z is independently a value of from about 1 to about 120, preferably from about 7 to about 100, and more preferably from about 7 to about 100, where x+y+z is greater than about 20.

Examples of the multi-polypropylene glycol-chain segment polymer of Formula IV which is especially useful herein includes polyoxypropylene glyceryl ether (n=1, R′=H, b=0, c and d=1, e=1, and x, y, and z independently indicate the degree of polymerization of their respective polypropylene glycol-chain segments; available as New Pol GP-4000, from Sanyo Kasei, Osaka, Japan), polypropylene trimethylol propane (n=1, R′=C₂H₅, b=1, c and d=1, e=1, and x, y, and z independently indicate the degree of polymerization of their respective polypropylene glycol-chain segments), polyoxypropylene sorbitol (n=4, each R′=H, b=0, c and d=1, each e=1, and y, z, and each x independently indicate the degree of polymerization of their respective polypropylene glycol-chain segments; available as New Pol SP-4000, from Sanyo Kasei, Osaka, Japan), and PPG-10 butanediol (n=0, c and d=2, and y+z=10; available as Probutyl DB-10, from Croda, Inc., of Parsippany, N.J., U.S.A.).

In a preferred embodiment, one or more of the propylene repeating groups in the polypropylene glycol is an isopropyl oxide repeating group. More preferably one or more of the propylene oxide repeating groups of the polypropylene glycol of Formula III and/or the polypropylene glycol of Formula IV is an isopropyl oxide repeating group. Even more preferably, substantially all of the propylene oxide repeating groups of the polypropylene glycol of Formula III and/or the polypropylene glycol of Formula IV are isopropyl oxide repeating groups. Accordingly, a highly preferred single-polypropylene glycol-chain segment polymer has the formula:

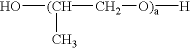

wherein a is defined as described above for Formula III. Similarly, a highly preferred multi-polypropylene glycol-chain segment polymer has the formula:

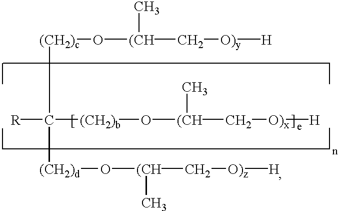

wherein n, R″, b, c, d, e, x, y, and z are defined as above, for Formula IV. It is recognized that the isopropyl oxide repeating groups may also correspond either alone, or in combination with the above depicted, to:

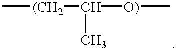

The polypropylene glycol useful herein is readily available from, for example, Sanyo Kasei (Osaka, Japan) as New pol PP-2000, New pol PP-4000, New pol GP-4000, and New pol SP-4000, from Dow Chemicals (Midland, Mich., USA), from Calgon Chemical, Inc. (Skokie, Ill., USA), from Arco Chemical Co. (Newton Square Pa., USA), from Witco Chemicals Corp. (Greenwich, Conn., USA), and from PPG Specialty Chemicals (Gurnee, Ill., USA).

Polyethylene Glycol

Preferably, the composition B of present invention may further comprise a polyethylene glycol having the formula:

H(OCH₂CH₂)ₙ —OH

wherein n has an average value of from 2,000 to 14,000, preferably from about 5,000 to about 9,000, more preferably from about 6,000 to about 8,000.

The polyethylene glycol can be included in the composition B at a level by weight of, preferably from about 0.1% to about 10%, more preferably from about 0.25% to about 6%.

The polyethylene glycol can be also included in the composition A at a level by weight of, preferably from about 0.1% to about 10%, more preferably from about 0.25% to about 6%.

The polyethylene glycol described above is also known as a polyethylene oxide, and polyoxyethylene. Polyethylene glycols useful herein that are especially preferred are PEG-2M wherein n has an average value of about 2,000 (PEG-2M is also known as Polyox WSR® N-10 from Union Carbide and as PEG-2,000); PEG-5M wherein n has an average value of about 5,000 (PEG-5M is also known as Polyox WSR® N-35 and as Polyox WSR® N-80, both from Union Carbide and as PEG-5,000 and Polyethylene Glycol 300,000); PEG-7M wherein n has an average value of about 7,000 (PEG-7M is also known as Polyox WSR® N-750 from Union Carbide); PEG-9M wherein n has an average value of about 9,000 (PEG-9M is also known as Polyox WSR® N-3333 from Union Carbide); and PEG-14M wherein n has an average value of about 14,000 (PEG-14M is also known as Polyox WSR® N-3000 from Union Carbide).

Preferred Hair Conditioning Compositions

The compositions of the present invention can be in the form of rinse-off products or leave-on products, and can be in the form of emulsion, cream, gel, spray or, mousse.

The compositions of the present invention have a suitable viscosity, preferably from about 1,000 mm²S⁻¹ to about 100,000 mm²S⁻¹, more preferably from about 2,000 mm²S⁻¹ to about 50,000 mm²S⁻¹. The viscosity herein can be suitably measured at 2.0s⁻¹ of shear rate after 1 minute of rotation.

In one preferred embodiment of the present invention, the hair conditioning composition comprises by weight:

(a) from about 0.1 % to about 15%, preferably from about 1% to about 10% of a high melting point fatty compound, preferably, the high melting point fatty compound selected from the group consisting of cetyl alcohol, stearyl alcohol, behenyl alcohol, and mixtures thereof;

(b) from about 0.1% to about 10%, preferably from about 0.5% to about 3% of an amidoamine having the following general formula:

R¹ CONH (CH₂)ₘ N (R²)₂

wherein R¹ is a residue of C₁₁ to C₂₄ fatty acids, R² is a C₁ to C₄ alkyl, and m is an integer from 1 to 4, preferably, the amidoamine selected from the group consisting of stearamidopropyl dimethylamine, stearamidoethyl diethylamine, and mixtures thereof;

(c) an acid selected from the group consisting of λ-glutamic acid, lactic acid, hydrochloric acid, malic acid, succinic acid, acetic acid, fumaric acid, λ-glutamic acid hydrochloride, tartaric acid, and mixtures thereof, at a level such that the mole ratio of amidoamine to acid is from about 1:0.3 to about 1:1, preferably, λ-Glutamic acid at a level such that the mole ratio of amidoamine to acid is from about 1:0.5 to about 1:0.9; and

(d) an aqueous carrier;

This composition may further contain a silicone compound at a level by weight of from about 0.1% to about 10%.

In another preferred embodiment of the present invention, the hair conditioning composition comprises by weight:

(a) from about 0.1% to about 15%, preferably from about 1% to about 10% of a high melting point fatty compound having a melting point of 25° C. or higher;

(b) from about 0.1% to about 10%, preferably from about 0.25% to about 5% of a cationic conditioning agent;

(c) an aqueous carrier; and

(d) from about 0.1% to about 10%, preferably from about 0.25% to about 6% of a polypropylene glycol.

This composition may further contain a low melting point oil having a melting point of less than 25° C. at a level by weight of from about 0.1% to about 10%, preferably from about 0.25% to about 6%, more preferably from about 0.3% to about 3%.

In another preferred embodiment of the present invention, the hair conditioning composition comprises by weight:

(a) from about 0.1% to about 15%, preferably from about 0.25% to about 5% of a high melting point fatty compound having a melting point of 25° C. or higher;

(b) from about 0.1% to about 10%, preferably from about 0.25% to about 5% of a cationic conditioning agent;

(c) from about 0.1% to about 10%, preferably from about 0.25% to about 6% of a low melting point oil having a melting point of less than 25° C., preferably, the low melting point oil being an unsaturated oil;

(d) an aqueous carrier; and

(e) from about 0.1% to about 10%, preferably from about 0.25% to about 6% of a polyethylene glycol.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, a mixture of Polysorbate 60 and Cetearyl Alcohol with tradename Polawax NF available from Croda Chemicals, glycerylmonostearate available from Stepan Chemicals, hydroxyethyl cellulose available from Aqualon, 3-pyridinecarboxy acid amide (niacinamide), hydrolysed keratin, proteins, plant extracts, and nutrients; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate, antidandruff agents such as zinc pyridinethione, and salicylic acid; and optical brighteners, for example polystyrylstilbenes, triazinstilbenes, hydroxycoumarins, aminocoumarins, triazoles, pyrazolines, oxazoles, pyrenes, porphyrins, imidazoles, and mixtures thereof.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Hair Conditioning Compositions

| Components | Comp. 1 | Comp. 2 | Comp. 3 |
|---|---|---|---|
| Cetyl Alcohol *1 | 2.0 | 2.5 | 2.0 |
| Stearyl Alcohol *2 | 3.6 | 4.5 | 3.6 |
| Stearamidopropyl Dimethylamine *3 | 1.6 | 2.0 | 1 .6 |
| l-Glutamic acid *4 | 0.412 | 0.64 | 0.412 |
| Mica *5 | — | — | 0.5 |
| Silica *6 | — | 1.0 | 1.0 |
| Silicone Blend *7 | 3.36 | 4.37 | 3.36 |
| Perfume | 0.4 | 0.4 | 0.4 |
| Benzyl alcohol | 0.4 | 0.4 | 0.4 |
| EDTA | 0.1 | 0.1 | 0.1 |
| Kathon CG *8 | 0.0005 | 0.0005 | 0.0005 |
| Sodium Chloride | 0.01 | 0.01 | 0.01 |
| 3-pyridinecarboxy acid amide | 0.05 | 0.05 | 0.05 |
| dl-Alpha tocopherol acetate | 0.05 | 0.05 | 0.05 |
| Hydrolyzed collagen *9 | 0.01 | 0.01 | 0.01 |
| Panthenol *10 | 0.05 | 0.05 | 0.05 |
| Panthenyl Ethyl Ether *11 | 0.05 | 0.05 | 0.05 |
| Octyl methoxycinnamate | 0.09 | 0.09 | 0.09 |
| Benzophenone-3 | 0.09 | 0.09 | 0.09 |

| | |
|---|---|
| Citric Acid | amount necessary to adjust pH 3-7 |
| Deionized Water | q.s. to 100% |

| Components | Comp. 4 | Comp. 5 |
|---|---|---|
| Cetyl Alcohol *1 | 2.6 | 2.0 |
| Stearyl Alcohol *2 | 4.6 | 3.6 |
| Stearamidopropyl Dimethylamine *3 | 1.8 | 1.6 |
| l-Glutamic acid *4 | 0.6 | 0.5 |
| Pentaerythritol Tetraisostearate *13 | 1.0 | 0.5 |
| Polypropylene Glycol *20 | 4.5 | 4.0 |
| Silica *6 | — | 1.0 |
| Silicone Blend *7 | 2.5 | 4.2 |
| Perfume | 0.4 | 0.4 |
| Benzyl alcohol | 0.4 | 0.4 |
| EDTA | 0.1 | 0.1 |
| Kathon CG *8 | 0.0005 | 0.0005 |
| Sodium Chloride | 0.01 | 0.01 |
| 3-pyridinecarboxy acid amide | 0.05 | 0.05 |
| dl-Alpha tocopherol acetate | 0.05 | 0.05 |
| Hydrolyzed collagen *9 | 0.01 | 0.01 |
| Panthenol *10 | 0.05 | 0.05 |
| Panthenyl Ethyl Ether *11 | 0.05 | 0.05 |
| Octyl methoxycinnamate | 0.09 | 0.09 |
| Benzophenone-3 | 0.09 | 0.09 |
| Citric Acid amount | necessary to adjust | |
| | pH 3-7 | |
| Deionized Water | g.s. to 100% | |
| Components | Comp. 6 | Comp. 7 |
| Cetyl Alcohol *1 | 0.96 | 1.2 |
| Stearyl Alcohol *2 | 0.64 | 0.8 |
| Stearamidopropyl Dimethylamine *3 | 1.0 | |
| Ditallow dimethyl ammonium chloride | 0.75 | 0.64 |
| *12 | | |
| Pentaerythritol Tetraisostearate *13 | 0.5 | |
| Pentaerythritol Tetraoleate *14 | | 0.2 |
| Oleyl alcohol *15 | | 0.25 |
| Trimethylolpropane Triisostearate *16 | | 0.25 |
| PEG 2M *17 | 0.5 | 0.5 |
| Polysorbate 60 *18 | 0.25 | 0.25 |
| Cetearyl Alcohol *18 | 0.25 | 0.25 |
| Glycerylmonostearate *19 | 0.25 | 0.25 |
| Silica *6 | 1.0 | 1.0 |
| Silicone Blend *7 | 4.2 | 4.2 |
| Perfume | 0.4 | 0.4 |
| Benzyl alcohol | 0.4 | 0.4 |
| EDTA | 0.1 | 0.1 |
| Kathon CG *8 | 0.0005 | 0.0005 |
| Sodium Chloride | 0.01 | 0.01 |
| 3-pyridinecarboxy acid amide | 0.05 | 0.05 |
| dl-Alpha tocopherol acetate | 0.05 | 0.05 |
| Hydrolyzed collagen *9 | 0.01 | 0.01 |
| Panthenol *10 | 0.05 | 0.05 |
| Panthenyl Ethyl Ether *11 | 0.05 | 0.05 |
| Octyl methoxycinnamate | 0.09 | 0.09 |
| Benzophenone-3 | 0.09 | 0.09 |

| | |
|---|---|
| Citric Acid | amount necessary to adjust |
| | pH 3-7 |
| Deionized Water | q.s. to 100% |

Definitions of Components
*1 Cetyl Alcohol: Konol series available from Shin Nihon Rika.
*2 Stearyl Alcohol: Konol series available from Shin Nihon Rika.
*3 Stearamidopropyl Dimethylamine: SAPDMA available from Inolex.
*4 l-Glutamic acid: l-Glutamic acid (cosmetic grade) available from Ajinomoto.
*5 Mica: Mearlmica CF available from Mearl.
*6 Silica: Neosil CBT 60 having an average particle size of 250-400 μm
*7 Silicone Blend: SE 76 available from General Electric
*8 Kathon OG: Methylchloroisothiazolinone and Methylisothiazolinone available from Rohm & Haas.
*9 Hydrolyzed collagen: Peptein 2000 available from Hormel.
*10 Panthenol: available from Roche.
*11 Panthenyl Ethyl Ether: available from Roche.
*12 Ditallow dimethyl ammonium chloride: Available from Witco Chemicals.
*13 Pentaerythritol Tetraisostearate: KAK PTI obtained by Kokyu alcohol.
*14 Pentaerythritol Tetraoleate: Available from Shin NihonRika.
*15 Oleyl alcohol: Available from New Japan Chemical.
*16 Trimethylolpropane Triisostearate: KAK TTI obtained by Kokyu alcohol.
*17 PEG-2M: Polyox obtained by Union Carbide.
*18 Polysorbate 60, Cetearyl Alcohol: mixture sold as Polawax NF obtained by Croda Chemicals.
*19 Glycerylmonostearate: Available from Stepan Chemicals.
*20 Polypropylene Glycol: PP2000 available from Sanyo Kasei.

Method of Preparation

The hair conditioning compositions of Compositions 1 through 7 as shown above can be prepared by any conventional method well known in the art. They are suitably made as follows: When included in the composition, polymeric materials such as polypropylene glycol are dispersed in water at room temperature to make a polymer solution, and heated up to above 70° C. Amidoamine and acid, and when present, other cationic surfactants, ester oil of low melting point oil are added in the solution with agitation. Then high melting point fatty compound, and when present, other low melting point oils and benzyl alcohol are also added in the solution with agitation. The mixture thus obtained is cooled down to below 60° C., and the remaining components such as silicone compound, and menthol are added with agitation, and further cooled down to about 30° C. Then particles such as mica and silica, if included, are added and mixed.

A triblender and/or mill can be used in each step, if necessary to disperse the materials.

### Example 1

Hair care kit A comprises 20 g of the hair conditioning composition 1 as a hair care composition, and 1.3 g of calcium oxide as a heat generating agent. By mixing the hair conditioning composition with the calcium oxide, the hair conditioning composition is warmed to a peak temperature of about 55° C.

### Example 2

Hair care kit A comprises 50 g of the hair conditioning composition 2 as a hair care composition, and 7 g of magnesium sulfate as a heat generating agent. By mixing the hair conditioning composition with the magnesium sulfate, the hair conditioning composition is warmed to a peak temperature of about 57° C.

### Example 3

Hair care kit A comprises 20 g of the hair conditioning composition 4 as a hair care composition, 1.3 g of magnesium as a heat generating agent, and 2.6 g of citric acid as a reaction control agent. By mixing the hair conditioning composition with the calcium oxide and citric acid, the hair conditioning composition is warmed to a peak temperature of about 65° C.

### Example 4

Hair care kit A comprises 50 g of the hair conditioning composition 7 as a hair care composition, 3.3 g of calcium oxide and 3.3 g of magnesium as heat generating agents, and 6.66 g of citric acid as a reaction control agent. By mixing the hair conditioning composition with the calcium oxide, magnesium, and citric acid, the hair conditioning composition is warmed to a peak temperature of about 80° C.

### Example 5

Hair care kit B using 50 g of the hair conditioning composition 1 as a hair care composition, comprises 3.3 g of calcium oxide and 3.3 g of magnesium as heat generating agents, and 13.3 g of citric acid as a reaction control agent, and water as a reacting means, wherein the calcium oxide, magnesium, and citric acid are dispersed in glycerin. By mixing calcium oxide, magnesium, and citric acid with water, heat generating reaction starts, the heat caused by the reaction is conducted to the hair conditioning composition, and the hair conditioning composition is warmed to a peak temperature of about 55° C.

### Example 6

Hair care kit B of EXAMPLE 5 using the hair conditioning composition 3 as a hair care composition instead of the hair conditioning composition 1. By mixing calcium oxide, magnesium, and citric acid with water, heat generating reaction starts, the heat caused by the reaction is conducted to the hair conditioning composition, and the hair conditioning composition is warmed to a peak temperature of about 55° C.

### Example 7

Hair care kit B using 50 g of the hair conditioning composition 5 as a hair care composition, comprises sodium acetate trihydrate as heat generating agents, and a stimulating switch as a reacting means. By turning on the stimulating switch, heat generating reaction starts, the heat caused by the reaction is conducted to the hair conditioning composition, and the hair conditioning composition is warmed to a peak temperature of about 43° C.

### Example 8

Hair care kit B of EXAMPLE 7 using the hair conditioning composition 6 as a hair care composition instead of the hair conditioning composition 5. By turning on the stimulating switch, heat generating reaction starts, the heat caused by the reaction is conducted to the hair conditioning composition, and the hair conditioning composition is warmed to a peak temperature of about 43° C.

### Example 9

Heating device which is shaped as a container and uses comprises 3.3 g of calcium oxide and 3.3 g of magnesium as heat generating agents, and 13.3 g of citric acid as a reaction control agent, wherein the calcium oxide, magnesium, and citric acid are dispersed in glycerin. By mixing water with calcium oxide, magnesium, and citric acid which are dispersed in glycerin, heat generating reaction starts. This device provides a peak temperature of above 55° C., and sustains a temperature of above 40° C. for more than 20 minutes.

### Example 10

Heating device which is shaped as a cap and uses a comprises sodium acetate trihydrate as heat generating agents, and a stimulating switch as a reacting means. By turning on the stimulating switch, heat generating reaction starts. This device provides a peak temperature of above 55° C., and sustains a temperature of above 40° C. for more than 20 minutes.

The embodiments disclosed herein have many advantages. For example, hair care compositions warmed by the hair care kits and heating devices of the present invention, can provide enhanced efficacy, i.e., can provide improved benefits. For example, warmed hair conditioning compositions can provide improved hair conditioning benefits such as moisturized feel, softness, and static control to the hair, due to improved penetration of ingredients.

It is understood that the examples and embodiments described herein are for illustrative purposes only and that various modifications or changes in light thereof will be suggested to one skilled in the art without departing from its spirit and scope.

What is claimed is:

1. A hair care kit comprising an aqueous hair care composition and a heat generating agent which generates a heat by mixing with the aqueous hair care composition, wherein the heat generating agent is isolated from the aqueous hair care composition.

2. The hair care kit according to claim 1, wherein the aqueous hair care composition is packed into a first container, the heat generating agent is packed into a second container, and the first and second containers are isolated from each other.

3. The hair care kit according to claim 1, wherein the aqueous hair care composition and the heat generating agent are packed into the same container comprising two compartments, the aqueous hair care composition is packed into a first compartment, the heat generating agent is packed into a second compartment.

4. The hair care kit according to claim 3, wherein the first and second compartments are isolated by a breakable partition.

5. The hair care kit according to claim 3, wherein the first and second compartments are isolated with each other by a breakable partition, the first and second compartments so positioned that the first or second compartment is encompassed with the other.

6. The hair care kit according to claim 1, wherein the heat generating agent is selected from the group consisting of the combination of iron, active carbon, and chloride, the combination of iron and potassium peroxodisulfate, calcium oxide, magnesium oxide, magnesium sulfate, calcium chloride, magnesium, magnesium chloride, iron (II) chloride, iron (III) chloride, zeolite, polyhydric alcohol, and mixtures thereof.

7. The hair care kit according to claim 6, wherein the heat generating agent is dispersed in an inert carrier.

8. The hair care kit according to claim 1 further comprising a reaction control agent.

9. The hair care kit according to claim 1 further comprising a heat reserving material.

10. The hair care kit according to claim 1, wherein the heat generating agent is mixed with the aqueous hair care composition before the aqueous hair care composition is applied to the hair.

11. The hair care kit according to claim 1 which is used for warming the aqueous hair care composition before the aqueous hair care composition is applied to the hair.

12. The hair care kit according to claim 11, wherein the aqueous hair care composition is warmed to a temperature of from about 25° C. to about 80° C.

13. The hair care kit according to claim 1, wherein the aqueous hair care composition is selected from the group consisting of an aqueous hair shampoo composition, an aqueous hair styling composition, an aqueous hair conditioning composition, an aqueous hair color composition, an aqueous hair growth composition, and mixtures thereof.

14. The hair care kit according to claim 13, wherein the aqueous hair care composition is an aqueous hair conditioning composition.

15. The hair care kit according to claim 14, wherein the aqueous hair conditioning composition comprises by weight:
(a) from about 0.1% to about 15% of a high melting point fatty compound;
(b) from about 0.1% to about 10% of an amidoamine having the following general formula:
R¹ CONH (CH₂)ₘ N (R²)₂
wherein R¹ is a residue of C₁₁ to C₂₄ fatty acids, R² is a C₁ to C₄ alkyl, and m is an integer from 1 to 4;
(c) an acid selected from the group consisting of λ-glutamic acid, lactic acid, hydrochloric acid, malic acid, succinic acid, acetic acid, fumaric acid, λ-glutamic acid hydrochloride, tartaric acid, and mixtures thereof, at a level such that the mole ratio of amidoamine to acid is from about 1:0.3 to about 1:1; and
(d) an aqueous carrier.

16. The hair care kit according to claim 15, wherein the aqueous hair conditioning composition further comprises by weight from about 0.1% to about 10% by weight of a silicone compound.

17. The hair care kit according to claim 15, wherein the aqueous hair conditioning composition further comprises by weight from about 0.1% to about 10% of a polypropylene glycol.

18. The hair care kit according to claim 15, wherein the aqueous hair conditioning composition further comprises by weight from about 0.01 % to about 10% of a particle.

19. The hair care kit according to claim 15, wherein the aqueous hair conditioning composition comprises by weight:
(a) from about 1% to about 10% of the high melting point fatty compound selected from the group consisting of cetyl alcohol, stearyl alcohol, behenyl alcohol, and mixtures thereof;
(b) from about 0.5% to about 3% of the amidoamine selected from the group consisting of stearamidopropyl dimethylamine, stearamidoethyl diethylamine, and mixtures thereof;
(c) λ-Glutamic acid at a level such that the mole ratio of amidoamine to acid is from about 1:0.5 to about 1:0.9; and
(d) an aqueous carrier.

20. The hair care kit according to claim 14, wherein the aqueous hair conditioning composition comprises by weight:
(a) from about 0.1% to about 15% of a high melting point fatty compound having a melting point of 25° C. or higher;
(b) from about 0.1% to about 10% of a cationic conditioning agent;
(c) from about 0.1% to about 10% of a low melting point oil having a melting point of less than 25° C.; and
(d) an aqueous carrier.

21. The hair care kit according to claim 20, wherein the low melting point oil is an unsaturated fatty alcohol.

22. The hair care kit according to claim 20, wherein the low melting point oil is selected from the group consisting of:
(a) pentaerythritol ester oils having a molecular weight of at least about 800, and having the following formula: 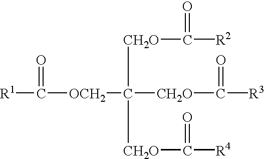
wherein R¹, R², R³, and R⁴, independently, are branched, straight, saturated, or unsaturated alkyl, aryl, and alkylaryl groups having from 1 to about 30 carbons;
(b) trimethylol ester oils having a molecular weight of at least about 800, and having the following formula: 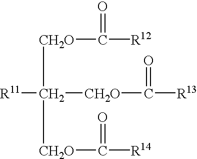
wherein R¹¹ is an alkyl group having from 1 to about 30 carbons, and R¹², R¹³, and R¹⁴, independently, are branched, straight, saturated, or unsaturated alkyl, aryl, and alkylaryl groups having from 1 to about 30 carbons;
(c) poly α-olefin oils derived from 1-alkene monomers having from about 6 to about 16 carbons, the poly α-olefin oils having a viscosity of from about 1 to about 35,000 cst, a molecular weight of from about 200 to about 60,000, and a polydispersity of no more than about 3;
(d) citrate ester oils having a molecular weight of at least about 500, and having the following formula: 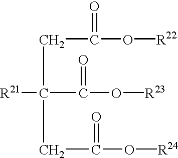
wherein R²¹ is OH or CH₃COO, and R²², R²³, and R²⁴, independently, are branched, straight, saturated, or unsaturated alkyl, aryl, and alkylaryl groups having from 1 to about 30 carbons;
(e) glyceryl ester oils having a molecular weight of at least about 500, and having the following formula: 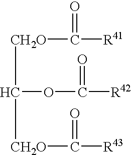
wherein R⁴¹, R⁴², and R⁴³, independently, are branched, straight, saturated, or unsaturated alkyl, aryl, and alkylaryl groups having from 1 to about 30 carbons; and mixtures thereof.

23. The hair care kit according to claim 20, wherein the aqueous hair conditioning composition further comprises by weight from about 0.1% to about 10% of a polyethylene glycol having the formula:
H(OCH₂CH₂)ₙ —OH
wherein n has an average value of from 2,000 to 14,000.

24. The hair care kit according to claim 20, wherein the aqueous hair conditioning composition further comprises by weight from about 0.01 % to about 10% of a particle.

25. A hair care kit comprising:
(i) a hair care composition;
(ii) a heat generating agent which generates a heat by reaction with a reacting means; and
(iii) a reacting means; wherein the hair care composition, the heat generating agent, and the heat generating support agent are isolated respectively.

26. The hair care kit according to claim 25, wherein the hair care composition, the heat generating agent, and the reacting means are packed into the same container comprising three compartments, the hair care composition is packed into a first compartment, the heat generating agent is packed into a second compartment, and the reacting means is packed into a third compartment.

27. The hair care kit according to claim 26, wherein the second and third compartments are isolated by a breakable partition.

28. The hair care kit according to claim 26, wherein the second and third compartments are isolated with each other by a breakable partition, the second and third compartments so positioned that the second compartment is encompassed with the third compartment.

29. The hair care kit according to claim 26, wherein the second and third compartments are isolated with each other by a breakable partition, the second and third compartments so positioned that the third compartment is encompassed with the second compartment.

30. The hair care kit according to claim 25, wherein the first compartment is isolated from the second and third compartments, by a non-breakable and heat conductive partition.

31. The hair care kit according to claim 25, wherein the heat generating agent is selected from the group consisting of the combination of iron, active carbon, and chloride, the combination of iron and potassium peroxodisulfate, calcium oxide, magnesium oxide, magnesium sulfate, calcium chloride, magnesium, magnesium chloride, iron (II) chloride, iron (III) chloride, zeolite, polyhydric alcohol, supercooled liquid, and mixtures thereof.

32. The hair care kit according to claim 31, wherein the heat generating agent is dispersed in an inert carrier.

33. The hair care kit according to claim 25, wherein the reacting means is selected from the group consisting of water, aqueous solution, aqueous composition, and mixtures thereof.

34. The hair care kit according to claim 25 further comprising a reaction control agent.

35. The hair care kit according to claim 25 further comprising a heat reserving material.

36. The hair care kit according to claim 25, wherein the heat generating agent is mixed with the reacting means before the hair care composition is applied to the hair.

37. The hair care kit according to claim 25 which is used for warming the hair care composition before the hair care composition is applied to the hair.

38. The hair care kit according to claim 25, wherein the hair care composition is warmed to a temperature of from about 25° C. to about 80° C.

39. The hair care kit according to claim 25, wherein the hair conditioning composition is selected from the group consisting of a hair shampoo composition, a hair styling composition, a hair conditioning composition, a hair color composition, a hair growth composition, and mixtures thereof.

40. The hair care kit according to claim 39, wherein the hair conditioning composition is a hair conditioning composition.

41. The hair care kit according to claim 40, wherein hair conditioning composition comprises by weight:
(a) from about 0.1% to about 15% of a high melting point fatty compound;
(b) from about 0.1% to about 10% of an amidoamine having the following general formula:
R¹ CONH (CH²)ₘ N (R²)₂
wherein R¹ is a residue of C₁₁ to C₂₄ fatty acids, R² is a C₁ to C₄ alkyl, and m is an integer from 1 to 4;
(c) an acid selected from the group consisting of λ-glutamic acid, lactic acid, hydrochloric acid, malic acid, succinic acid, acetic acid, fumaric acid, λ-glutamic acid hydrochloride, tartaric acid, and mixtures thereof, at a level such that the mole ratio of amidoamine to acid is from about 1:0.3 to about 1:1; and
(d) an aqueous carrier.

42. The hair care kit according to claim 41, wherein the hair conditioning composition further comprises by weight from about 0.1% to about 10% by weight of a silicone compound.

43. The hair care kit according to claim 41, wherein the hair conditioning composition further comprises by weight from about 0.1% to about 10% of a polypropylene glycol.

44. The hair care kit according to claim 41, wherein the hair conditioning composition further comprises by weight from about 0.01 % to about 10% of a particle.

45. The hair care kit according to claim 41, wherein the hair conditioning composition comprises by weight:
(a) from about 1% to about 10% of the high melting point fatty compound selected from the group consisting of cetyl alcohol, stearyl alcohol, behenyl alcohol, and mixtures thereof;
(b) from about 0.5% to about 3% of the amidoamine selected from the group consisting of stearamidopropyl dimethylamine, stearamidoethyl diethylamine, and mixtures thereof;
(c) λ-Glutamic acid at a level such that the mole ratio of amidoamine to acid is from about 1:0.5 to about 1:0.9; and
(d) an aqueous carrier.

46. The hair care kit according to claim 40, wherein the hair care composition is a hair conditioning composition comprising by weight:
(a) from about 0.1% to about 15% of a high melting point fatty compound having a melting point of 25° C. or higher;
(b) from about 0.1% to about 10% of a cationic conditioning agent;
(c) from about 0.1% to about 10% of a low melting point oil having a melting point of less than 25° C.; and
(d) an aqueous carrier.

47. The hair care kit according to claim 46, wherein the low melting point oil is an unsaturated fatty alcohol.

48. The hair care kit according to claim 46, wherein the low melting point oil is selected from the group consisting of:
(a) pentaerythritol ester oils having a molecular weight of at least about 800, and having the following formula: 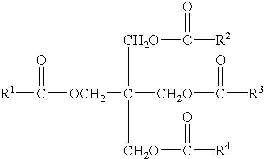
wherein R¹, R², R³, and R⁴, independently, are branched, straight, saturated, or unsaturated alkyl, aryl, and alkylaryl groups having from 1 to about 30 carbons;
(b) trimethylol ester oils having a molecular weight of at least about 800, and having the following formula: 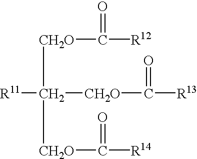
wherein R¹¹ is an alkyl group having from 1 to about 30 carbons, and R¹², R¹³, and R¹⁴, independently, are branched, straight, saturated, or unsaturated alkyl, aryl, and alkylaryl groups having from 1 to about 30 carbons;
(c) poly α-olefin oils derived from 1-alkene monomers having from about 6 to about 16 carbons, the poly α-olefin oils having a viscosity of from about 1 to about 35,000 cst, a molecular weight of from about 200 to about 60,000, and a polydispersity of no more than about 3;
(d) citrate ester oils having a molecular weight of at least about 500, and having the following formula: 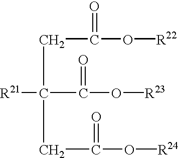
wherein R²¹ is OH or CH₃COO, and R²², R²³, and R²⁴, independently, are branched, straight, saturated, or unsaturated alkyl, aryl, and alkylaryl groups having from 1 to about 30 carbons;
(e) glyceryl ester oils having a molecular weight of at least about 500, and having the following formula: 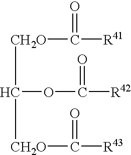
wherein R⁴¹, R⁴², and R⁴³, independently, are branched, straight, saturated, or unsaturated alkyl, aryl, and alkylaryl groups having from 1 to about 30 carbons; and mixtures thereof.

49. The hair care kit according to claim 46, wherein the hair conditioning composition further comprises by weight from about 0.1% to about 10% of a polyethylene glycol having the formula:
H(OCH₂CH₂)ₙ —OH
wherein n has an average value of from 2,000 to 14,000.

50. The hair care kit according to claim 46, wherein the hair conditioning composition further comprises by weight from about 0.01% to about 10% of a particle.

51. A heating device, used for warming a hair care composition, comprising a heating source.

52. The heating device according to claim 51 further comprising a non-breakable and heat conductive layer which covers the heating source.

53. The heating device according to claim 51, wherein the heating device is provided in the shape of a container suitable for receiving the hair care composition.

54. The heating device according to claim 51, wherein the heating device is used for warming hair care composition before the hair care composition is applied to the hair.

55. The heating device according to claim 51, wherein the heating device is provided in the shape suitable for covering the hair.

56. The heating device according to claim 55, wherein the heating device is provided in the shape of a cap.

57. The heating device according to claim 51, wherein the heating device is used for warming hair care composition after the hair care composition is applied to the hair.

58. The heating device according to claim 51, wherein the hair care composition is selected from the group consisting of a hair shampoo composition, a hair styling composition, and a hair conditioning composition, a hair color composition, a hair growth composition, and mixtures thereof.

59. The heating device according to claim 51, wherein the hair care composition is warmed to a temperature of from about 25° C. to about 80° C.

60. The heating device according to claim 51, wherein the heating source is selected from the group consisting of:
(i) a heat generating agent and a reacting means, wherein the heat generating agent generates a heat by reaction with the reacting means, and wherein the heat generating agent is isolated from the reacting means;
(ii) a heat reserving material;
(iii) a resistive heating system;
(iv) an electromagnetic induction heating system; and
(v) mixtures thereof.

61. The heating device according to claim 60, wherein the heating source comprises a heat generating agent and a reacting means, the heating source further comprising a first compartment which the heat generating agent is packed into, and a second compartment which the reacting means is packed into, the first and second compartments being isolated by a breakable partition.

62. The heating device according to claim 61, wherein the first and second compartments are isolated with each other by a breakable partition, the first and second compartments so positioned that the first or second compartment is encompassed with the other.

63. The heating device according to claim 60, wherein the heat generating agent is selected from the group consisting of the combination of iron, active carbon, and chloride, the combination of iron and potassium peroxodisulfate, calcium oxide, magnesium oxide, magnesium sulfate, calcium chloride, magnesium, magnesium chloride, iron (II) chloride, iron (III) chloride, zeolite, polyhydric alcohol, supercooled liquid, and mixtures thereof.

64. The heating device according to claim 63, wherein the heat generating agent is dispersed in an inert carrier.

65. The heating device according to claim 60, wherein the reacting means is selected from the group consisting of water, aqueous solution, aqueous composition, and mixtures thereof.

66. The heating device according to claim 60, wherein the heating source comprises a heat generating agent and a reacting means, and further comprises a reaction control agent.

67. The heating device according to claim 60, wherein the heat reserving material is selected from the group consisting of silica gel, carboxymethyl cellulose gel, phase-changing materials, and mixtures thereof.

68. The heating device according to claim 60, wherein the resistive heating system is selected from the group consisting of nichrome wire, ceramics, electrically conductive polymers, and mixtures thereof.
